(19) United States
(12) Patent Application Publication
(10) Pub. No.: US 2006/0074291 A1
(43) Pub. Date: Apr. 6, 2006

(54) Magnetic resonance imaging system and method

(75) Inventors: Christopher Judson Hardy, Schenectady, NY (US); William Thomas Dixon, Clifton Park, NY (US); Xuli Zong, Niskayuna, NY (US)

Correspondence Address: GENERAL ELECTRIC COMPANY;GLOBAL RESEARCH, PATENT DOCKET RM. BLDG. K1-4A59, NISKAYUNA, NY, 12309, US

(73) Assignee: General Electric Company

(21) Appl. No.: 10/955,630

(22) Filed: Sep. 30, 2004

(51) Int. Cl. A61B 5/05

(52) U.S. Cl. 600/410

(57) ABSTRACT

A magnetic resonance imaging (MRI) system using a double inversion recovery (DIR) imaging pulse sequence for acquiring black-blood images for obtaining a first level of nulling of a blood signal from acquired imaging slices is provided. The MRI system comprises an image processor configured changing a respective time order of the imaging slices at a selected point or points during the pulse sequence to obtain a second level of nulling of the residual blood signal from the imaging slices.

### BACKGROUND

The invention relates generally to magnetic resonance imaging (MRI) systems and more specifically to a method and system for acquiring images in MRI systems.

MRI pulse sequences such as the double-inversion-recovery fast-spin-echo (DIRFSE) pulse sequences have the useful property of nulling the signals from blood that is flowing through an imaging plane. The resulting black-blood images allow better visualization of vessel walls including any plaque components, myocardium, and the like, because of reduced competition with bright blood signals from neighboring pixels.

Typically, the DIRFSE pulse sequence employs, after an electrocardiogram (ECG) trigger, a nonselective inversion pulse and, at nearly the same time, a slice-selective inversion of a slice that incorporates the imaging slice. After a delay period (typically several hundred milliseconds) depending on the T₁ of blood, a fast spin echo (FSE) imaging sequence is played out.

The combination of the selective and nonselective inversion pulses produces essentially no net effect for static tissue in the plane (which is inverted by both pulses), resulting in a standard FSE-type image. However, most of the moving blood experiences only the nonselective inversion pulse, and so after the delay of T₁-null (when the recovering longitudinal magnetization is passing through zero) when the signals are read out, the blood signal is negligible. The few blood spins that experience both inversion pulses do not produce signals because the spins flow out of the imaging plane before the FSE portion of the pulse sequence is applied.

It is generally useful to acquire more than one slice within the T₁-null period as it improves imaging efficiency. Currently, the pulse sequences that are typically used for acquiring multiple slices are useful for producing T2-weighted contrast, but may not be used to generate T2* contrast, which is normally obtained with gradient-echo, bright-blood imaging. Gradient echo imaging is typically fast and T2* sensitive, however in arterial wall studies, FSE combine more readily with black blood.

Such pulse sequences are also not very efficient because FSE signals can only be read out at or near the T₁ null thus making the useful imaging window less than a hundred millisecond out of each heart beat. Some attempts have been made to acquire more than one slice within the T₁ null period, but they are constrained to operate within the narrow window.

Thus, there is a need for a method to increase the efficiency of multi-slice DIRFSE pulse sequences and to produce T2* contrast in DIRFSE pulse sequences while preserving their black-blood properties.

### BRIEF DESCRIPTION

Briefly, according to one aspect of the invention, a method for acquiring black-blood images using a magnetic resonance imaging (MRI) system and a double inversion recovery (DIR) imaging pulse sequence for obtaining a first level of nulling of the blood signal from acquired imaging slices is provided. The method comprises changing a respective time order of the imaging slices at a selected point or points during the pulse sequence to obtain a second level of nulling of the residual blood signal from the imaging slices.

In another embodiment, a magnetic resonance imaging (MRI) system using a double inversion recovery (DIR) imaging pulse sequence for obtaining a first level of nulling of the blood signal from acquired imaging slices is provided. The MR system comprises an image processor configured changing a respective time order of the imaging slices at a selected point or points during the pulse sequence to obtain a second level of nulling of the residual blood signal from the imaging slices.

In another embodiment, a double inversion recovery imaging pulse sequence for acquiring black-blood images for obtaining a first level of nulling of a blood signal from acquired imaging slices using a magnetic resonance imaging (MRI) system is provided. The DIRFSE pulse sequence comprises a double inversion recovery signal preparation component and an imaging component. The double inversion recovery signal preparation component includes a non-selective inversion pulse and a slab-selective inversion pulse, which slab covers a region encompassing a plurality of imaging slices. The imaging component includes the plurality of imaging slices whose signals are acquired over a range of times substantially centered on the inversion-null time of blood.

### DRAWINGS

These and other features, aspects, and advantages of the present invention will become better understood when the following detailed description is read with reference to the accompanying drawings in which like characters represent like parts throughout the drawings, wherein:

FIG. 1 is a block diagram of a magnetic resonance imaging system to which embodiments of the present invention are applicable;

FIG. 2 is a diagrammatical view of a double inversion recovery fast spin echo (DIRFSE) pulse sequence implemented according to the invention; and,

FIG. 3 illustrates a diagrammatical view of a multi-slice double inversion recovery fast spin echo (DIRFSE) pulse sequence implemented according to the invention.

### DETAILED DESCRIPTION

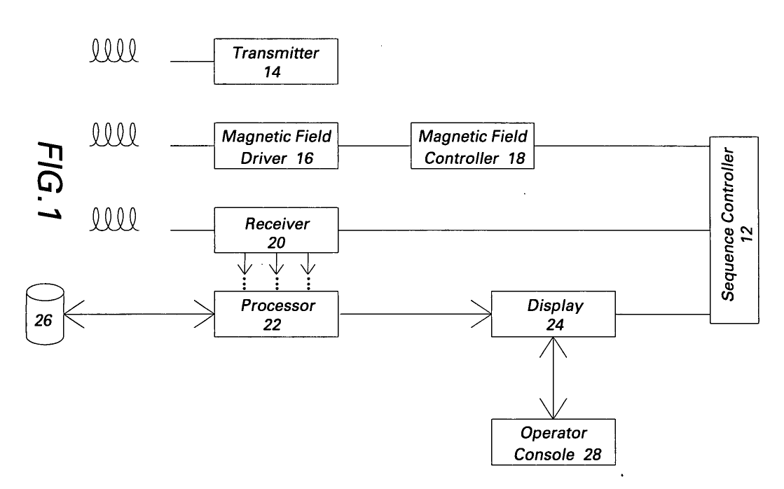

FIG. 1 is a block diagram of an exemplary magnetic resonance imaging (MRI) system for which embodiments of the present invention are applicable. The MR system could be, for example, a GE-Signa MR scanner available from GE Healthcare, which is adapted to perform the method of the present invention, although other systems could be used also.

As used herein, “adapted to”, “configured” and the like refer to devices in a system to allow the elements of the system to cooperate to provide a described effect; these terms also refer to operation capabilities of electrical or optical elements such as analog or digital computers or application specific devices (such as an application specific integrated circuit (ASIC)), amplifiers or the like that are programmed to provide an output in response to given input signals, and to mechanical devices for optically or electrically coupling components together

The MRI system 10 comprises a sequence controller 12 for controlling various components of the system, as is well-known, for detecting magnetic resonance signals from the part of an object being imaged. A transmitter 14 is configured for generating a radio frequency (RF) pulse to cause resonance. A magnetic field driver 16 is configured for driving a field gradient in a known manner and a magnetic field controller 18 coupled to the magnetic field driver 16 is configured for controlling the magnetic field.

Receiver 20 is configured for receiving and detecting magnetic resonance signals generated from the object. A processor 22 receives information from the receiver and is configured for performing image reconstruction and various calculations for system operation. The processor is coupled to display 24, which is configured for displaying images to an operator using the MRI system. The processor is also coupled to a storage 26 for storing detected signal data and reconstructed k-space data.

In a well-known manner, processor 22 is configured such that there is sufficient memory for storing measured data and reconstructed images. The memory is sufficient to store the whole of N-dimensional measured data as well as reconstructed data. Also in a well-known manner, an MR image is constructed from the image or k-space data corresponding to a predetermined plurality of applications of an MRI pulse sequence initiated by a RF pulse such as from transmitter 14 of FIG. 1. The image is updated by collecting image or k-space data from repetitive MRI pulse seqences. An MR image is reconstructed by performing a series of Fourier transforms along a set of orthogonal directions in k-space.

As a general description, magnetic resonance imaging (MRI) is a well-known imaging method in which magnetic moments are excited at specific nuclear spin precession frequencies that are proportional to the magnetic field occurring within the magnet of the MRI system. Spin is a fundamental property of nature, such as electrical charge or mass. As is well known, precession is a motion about an axis of a vector whose origin is fixed at the origin. The radio-frequency (RF) signals resulting from the precession of these spins are received typically using RF coils in the MRI system and are used to generate images of a volume of interest.

A pulse sequence is a selected series of RF pulses and/or magnetic field gradients applied to a spin system to produce a signal representative of some property of the spin system. Described herein are embodiments employing a double-inversion-recovery fast-spin-echo (DIRFSE) pulse sequence which can be used for nulling the signals from blood that is flowing through an imaging plane. The resulting images using DIRFSE are referred to herein as “black-blood images”. The resulting black-blood images allow better visualization of vessel walls including any plaque components, myocardium, etc., because of reduced competition with bright blood signals from neighboring pixels. The DIRFSE pulse sequence is adapted to rapidly create images characterized by T2* contrast.

The time constant that describes the return to equilibrium or specifically decay toward zero, of the transverse magnetization, Mxy, is called the spin-spin relaxation time, T2. T1 governs the rate of recovery of the longitudinal magnetization. T2* is the spin-spin relaxation time composed of contributions from molecular interactions and inhomogeneities in the magnetic field.

In the illustrated embodiment, the processor 22 configured to apply a double inversion recovery (DIR) imaging pulse sequence to acquire black blood images and to change a time order of the slices in the DIR imaging pulse sequence. A user using the MRI system may change in the time order of the slices.

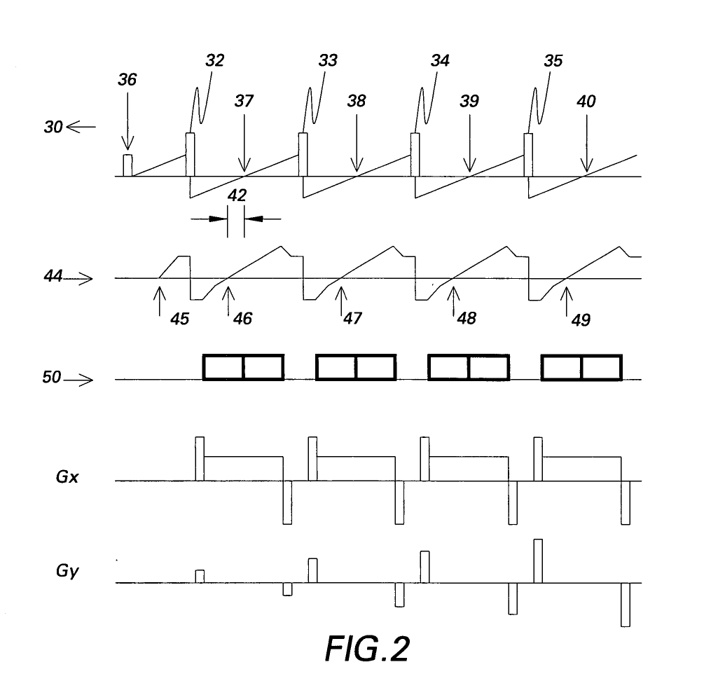

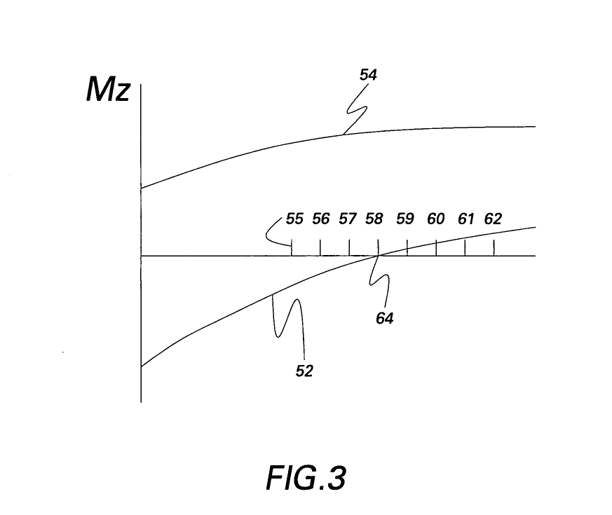

As will be described in further detail with reference to FIG. 2 and FIG. 3, the DIR imaging pulse sequence comprises a double inversion recovery signal-preparation component which includes a non-selective inversion pulse and a slab-selective inversion pulse. As is well-known, the DIR imaging pulse sequence is adapted to null blood signals from acquired image slices. The slab covers a region encompassing a plurality of imaging slices.

The DIRFSE pulse sequence further comprises an imaging component comprising a plurality of imaging slices having corresponding signals that are acquired over a range of times substantially centered on the inversion-null time of blood. The imaging component includes a fast spin echo (FSE) pulse sequence. In embodiments of the invention, the DIR imaging pulse sequence is used to obtain a first level of nulling of the residual blood signal from acquired image slices. After the DIRFSE pulse is applied, a respective time order of the image slices is changed at a selected point during acquisition to obtain a second level of nulling of the residual blood signal from the image slices.

In one embodiment, the time order is changed by reversing one half of averaged acquisitions relative to another of averaged acquisitions. As used herein, the term “averaged acquisitions” refers to the practice of applying a number of excitations and averaging the acquired image data, e.g. NEX image acquisition. The DIR pulse sequence comprises an even number of averaged acquisitions.

In another embodiment, the time order is changed by reversing a time order for signals acquired in a top half of k space relative to signals acquired in a bottom half of k space. This results in a phase twist in the residual blood signal relative to the signal from surrounding static tissue. The phase twist can be used to suppress the blood signal

Operator console 28 enables a user using the MRI system to interface with processor 22. The operator can adjust the time order of the slices, thus obtaining a desired T2* contrast. In one embodiment, increasing the pre-determined amount of offset results in a corresponding increase the T2* contrast

FIG. 2 is a diagrammatical view of a double inversion recovery fast spin echo (DIRFSE) pulse sequence implemented according to the invention. As described earlier, the DIRFSE pulse sequence 30 comprises a double-inversion recovery (DIR) component 32-35 followed by a fast spin echo (FSE) component represented by 36-40. The DIRFSE pulse sequence further comprises gradient echoes and spin echoes. The gradient echoes are represented by zero crossings on reference numeral 44 and the data acquisition cycle is represented by 50. Gx and Gy represent the pulse sequence transmitted by gradient coils.

As can be seen, the 90° FSE sequence 37-40 has been offset in time from its usual location illustrated by reference numeral 36 by a pre-determined amount ‘δ’ represented by reference numeral 42. An operator using the MRI system may provide the values of ‘δ’. By offsetting the spin echoes 37-40 from gradient echoes 46-49 by the predetermined amount ‘δ’, varying degrees of T2* contrast can be achieved.

In another embodiment, the gradient echoes represented generally by reference numeral 44 may be offsetted by ‘δ’ rather than the spin echo. In shifting the gradient echoes, the even and odd echoes are shifted in the same direction. The gradient echoes can be offset by inserting extra gradient lobes in the readout gradient Gₓ as shown in FIG. 2. In one specific embodiment, offsetting the gradient echo comprises changing a position in time of an excitation pulse relative to a refocusing pulse. In an alternate embodiment, offsetting the gradient echo comprises changing an area under a dephasing gradient pulse relative to the area under a rephasing gradient pulse.

FIG. 3 illustrates a diagrammatical view of a multi-slice double inversion recovery fast spin echo (DIRFSE) pulse sequence implemented according to the invention. In the illustrated embodiment, the DIRFSE pulse sequence is an example of an 8-slice sequence, the slices being represented by reference numerals 55-62. However, the technique is applicable to other slice sequences as well.

As shown in FIG. 3, the slices are acquired at times arranged in symmetric pairs about the blood null point 64. The slice examined at time 55 and the slice examined at time 62 form a symmetric pair about null point 64, similarly, time slots 56 and 61, time slots 57 and 60 and slots 58 and 59 also form symmetric pairs. Within each pair, the residual longitudinal magnetization from the blood signal 52, at each of the two times has roughly equal magnitude but opposite sign. However for the vessel wall 54 the longitudinal magnetization is roughly the same at the two times within each pair.

As described earlier, the processor is configured to modulate k-space acquisition to invert at least a portion of acquired slices representative of blood signals. In the illustrated example, the new order of the slices is 62-55. The new order indicates that the residual blood signals from each slice have the phase inverted in one half of k-space relative to the other half.

On applying a complex discrete Fast Fourier Transform to the signal from the blood is pushed into a quadrature (Q) component. If the image is then formed from the in-phase (I) component alone, the residual blood signal will be suppressed.

Typically, a phase roll across the image may exist. The inhomogeneity can be corrected for in manners known in the art, and the additional phase twist into the quadrature component for the blood caused by slice reordering can be preserved.

While only certain features of the invention have been illustrated and described herein, many modifications and changes will occur to those skilled in the art. It is, therefore, to be understood that the appended claims are intended to cover all such modifications and changes as fall within the true spirit of the invention.

1. A method for acquiring black-blood images using a magnetic resonance imaging (MRI) system and a double inversion recovery (DIR) imaging pulse sequence for obtaining a first level of nulling of a blood signal from acquired imaging slices, the method comprising:
changing a respective time order of the imaging slices at a selected point or points during the pulse sequence to obtain a second level of nulling of the residual blood signal from the imaging slices.

2. The method of claim 1, wherein the DIR imaging pulse sequence comprises a double inversion recovery signal-preparation component and an imaging component, wherein:
the double inversion recovery signal preparation component includes a non-selective inversion pulse and a slab-selective inversion pulse, which slab covers a region encompassing a plurality of imaging slices; and,
the imaging component includes the plurality of imaging slices whose signals are acquired over a range of times substantially centered on the inversion-null time of blood.

3. The method of claim 2, wherein the imaging component comprises a fast spin echo (FSE) pulse sequence.

4. The method of claim 1, wherein the DIR pulse sequence acquires an even number of averaged acquisitions, and wherein the time order of the slices for one half of the averaged acquisitions is reversed relative to another half.

5. The method of claim 1, wherein the time order of the slices is reversed for signals acquired in a top half of k space relative to signals acquired in a bottom half of k space resulting in a phase twist in the residual blood signal relative to the signal from surrounding static tissue.

6. The method of claim 5, wherein the phase twist is used to suppress the blood signal.

7. The method of claim 1, wherein the DIR imaging pulse sequence is configured to null signals from blood flowing through an imaging plane.

8. The method of claim 3, wherein the DIRFSE pulse sequence generates gradient echoes and spin echoes.

9. The method of claim 8, further comprising offsetting the gradient echoes in time relative to the corresponding spin echoes by a pre-determined amount of offset.

10. The method of claim 9, wherein the offsetting comprises changing a position in time of an excitation pulse relative to a refocusing pulse.

11. The method of claim 9, wherein the offsetting comprises changing an area under a dephasing gradient pulse relative to the area under a rephasing gradient pulse.

12. The method of claim 9, further comprising obtaining T2* contrast by adjusting the pre-determined amount of offset.

13. The method of claim 9, wherein a user operating the MRI system performs the adjusting of the pre-determined amount of offset.

14. The method of claim 13, wherein increasing the pre-determined amount of offset results in a corresponding increase in the T2* contrast.

15. A magnetic resonance imaging (NRI) system using a double inversion recovery (DIR) imaging pulse sequence for acquiring black-blood images, and the DIR imaging pulse sequence obtaining a first level of nulling of a blood signal from acquired imaging slices, the MRI system comprising:
an image processor configured for changing a respective time order of the imaging slices at a selected point or points during the pulse sequence to obtain a second level of nulling of the residual blood signal from the imaging slices.

16. The MRI system of claim 15, wherein the DIR imaging pulse sequence comprises a double inversion recovery signal-preparation component and an imaging component, wherein:
the double inversion recovery signal preparation component includes a non-selective inversion pulse and a slab-selective inversion pulse, which slab covers a region encompassing a plurality of imaging slices; and,
the imaging component includes the plurality of imaging slices whose signals are acquired over a range of times substantially centered on the inversion-null time of blood.

17. The MRI system of claim 16, wherein the imaging component comprises a fast spin echo (FSE) pulse sequence.

18. The MRI system of claim 16, wherein the DIR pulse sequence acquires an even number of averaged acquisitions, and wherein the time order of the slices for one half of the averaged acquisitions is reversed relative to another half.

19. The MRI system of claim 16, wherein the image processor is further configured to reverse the time order of the slices for signals acquired in a top half of k space relative to signals acquired in a bottom half of k space resulting in a phase twist in the residual blood signal relative to the signal from surrounding static tissue.

20. The MRI system of claim 19, wherein the phase twist is used to suppress the blood signal.

21. The MRI system of claim 17, wherein the DIRFSE pulse sequence generates gradient echoes and spin echoes.

22. The MRI system of claim 21, wherein the image processor is further configured to offset the gradient echoes in time relative to the corresponding spin echoes by a pre-determined amount of offset.

23. The MRI system of claim 22, wherein the image processor is configured to offset the gradient echoes in time relative to the corresponding spin echoes by the pre-determined amount of offset by changing a position in time of an excitation pulse relative to a refocusing pulse.

24. The MRI system of claim 22, wherein the image processor is configured to offset the gradient echoes in time relative to the corresponding spin echoes by the pre-determined amount of offset comprises changing an area under a dephasing gradient pulse relative to the area under a rephasing gradient pulse.

25. The MRI system of claim 22, wherein the image processor is further configured to obtain a T2* contrast by adjusting the pre-determined amount of offset.

26. The MRI system of claim 22, wherein a user operating the MRI system performs the adjusting of the pre-determined amount of offset.

27. The MRI system of claim 26, wherein the image processor is configured to increase the T2* contrast by a corresponding increase in the pre-determined amount of offset.

28. A double inversion recovery (DIR) pulse sequence for acquiring black-blood images for obtaining a first level of nulling of a blood signal from acquired imaging slices using an magnetic resonance imaging (MRI) system; the pulse sequence comprising:
a double inversion recovery signal-preparation component including a non-selective inversion pulse and a slab-selective inversion pulse, which slab covers a region encompassing a plurality of imaging slices;
an imaging component including the plurality of imaging slices whose signals are acquired over a range of times substantially centered on the inversion-null time of blood; and,
wherein a respective time order of the imaging slices is changed at a selected point or points during the pulse sequence to obtain a second level of nulling of the residual blood signal from the imaging slices.

29. The pulse sequence of claim 28, wherein the imaging component comprises a fast spin echo (FSE) pulse sequence.

30. The pulse sequence of claim 29, wherein the pulse sequence comprise gradient echoes and spin echoes.

31. The pulse sequence of claim 28, wherein the pulse sequence is configured to null signals from blood flowing through an imaging plane.
